(19) United States
(12) Patent Application Publication
(10) Pub. No.: US 2025/0098956 A1
(43) Pub. Date: Mar. 27, 2025

(54) A HEAD FOR THE GOLDMANN APPLANATION TONOMETER, A METHOD OF MEASURING AN INTRAOCULAR PRESSURE AND A METHOD OF IN VIVO MEASUREMENT OF A MODULUS OF ELASTICITY OF A CORNEA

(71) Applicant: IROMED GROUP S.R.L., Roma RM (IT)

(72) Inventors: Ciro CARUSO, Napoli NA (IT); Ciro COSTAGLIOLA, Napoli NA (IT)

(73) Assignee: IROMED GROUP S.R.L., Roma RM (IT)

(21) Appl. No.: 18/728,242

(22) PCT Filed: Dec. 30, 2022
(86) PCT No.: PCT/IB2022/062926

§ 371 Date: Jul. 11, 2024

(30) Foreign Application Priority Data
Jan. 11, 2022 (IT) 102022000000278

(51) Int. Cl. A61B 3/16

(52) U.S. Cl. CPC A61B 3/16

(57) ABSTRACT

By properly shaping the prisms of a Goldmann tonometer head, it becomes possible to accurately repeat two different pressure measurements with two different radii of the flattened corneal surface. From these two different measurements, with a method according to the present disclosure it is possible to obtain the value of the “true” intraocular pressure of a patient and therefore to correct the classic pressure measurement taken with the Goldmann tonometer. Furthermore, by knowing the central corneal thickness with any pachymeter and an average radius of curvature at the corneal apex with any keratometer, with another method according to the present disclosure it is possible to calculate in vivo the modulus of elasticity of the cornea of a patient using the two different pressure measurements.

### TECHNICAL FIELD

This disclosure relates to tonometers for measuring intraocular pressure and more particularly to a head for the Goldmann applanation tonometer, to a method for measuring the intraocular pressure of a human or animal eye, and to a method for in vivo measurement of the modulus of elasticity of a cornea.

### BACKGROUND

The measurement of intraocular pressure (IOP: IntraOcular Pressure) represents a fundamental moment of the routine ophthalmological examination, as the increase in its value represents the greatest risk factor for the onset of glaucoma and one of the fundamental parameters for the diagnosis of this disease, along with optic disc evaluation and visual field testing. Furthermore, the measurement of IOP provides useful indications for other ocular pathologies, such as uveitis, and in all patients undergoing eye surgery, and it is considered a routine examination to be performed in all ophthalmological visits.

The measurement of intraocular pressure can be performed with direct (manometry) or indirect (tonometry) methods. Ocular manometry is not used in clinical practice as it is more invasive, but is limited only to experimental use. Instead, ocular tonometry is the method most commonly used in clinical practice. It allows indirect measurement of intraocular pressure using instruments called tonometers, whose principle is based on the relationship between intraocular pressure and the force necessary to change the natural shape of the cornea.

In the following, reference will be made only to the “applanation” tonometers, which measure the force necessary to flatten a known surface area of the cornea or evaluate the size of the area flattened by a pre-established fixed force, as it is to them that the invention is applied.

The term “applanation tonometry”, in the following description and in the claims, is intended to indicate any tonometer which exploits the principles of applanation tonometry to determine the intraocular pressure.

Applanation tonometry is based on the fact that, to flatten a surface of area A of the cornea, an average force F is required which acts on the surface of area A in order to balance the intraocular pressure (IOP):

IOP(Intra Ocular Pressure)=F/A

It follows that the pressure inside an ideal sphere can be known by evaluating the width of the corneal area smoothed out by a constant force or by evaluating the force necessary to smooth out a known corneal area. Therefore, two different types of tonometers can be used for applanation tonometry: variable area tonometers and variable force tonometers.

The widest known and most used applanation tonometers in the world are those with variable force and undoubtedly the most famous of these is the Goldmann tonometer, now universally used, which represents the international standard for measuring IOP.

The Goldmann tonometer, represented in FIG. 1, comprises a smoothing element, consisting of a head typically made of transparent plastic or glass and containing a prism, joined by means of a sealing ring on a rod to a spiral spring. By means of a lateral graduated knob it is possible to vary the value of the force applied on the cornea, which is expressed in millimeters of mercury (mmHg) and is indicated by notches engraved on the knob itself.

The value of the flattened central cornea area is 3.06 mm² and this value, always constant, is set as the standard reference value.

The examination technique involves placing the instrument on a special base of a slit lamp, represented in FIG. 2. After corneal anesthesia, a coloring substance is applied to the patient's lower conjunctival arch on the anterior ocular surface, the coloring substance being a fluorescent substance that serves to make the edge of the flattened area more evident by observing with the cobalt blue light supplied with the slit lamp. As is known, identifying the edges of the flattened area with good precision is essential for a correct measurement of ocular pressure.

FIG. 3 is a perspective view of a conventional applanation tonometer head. It appears externally as an object with substantially rotational symmetry along a longitudinal axial direction and has an integral body defining:
at a first end along the longitudinal axial direction, an end portion 2 defining a flat corneal applanation surface S1, having a respective applanation radius, arranged on a first base plane of the end portion 2 orthogonal to a longitudinal axis of the tonometer head;
two semi-cylindrical prisms 3a and 3b integral with the end portion 2, each having:
   a respective lower semicircular base arranged along a respective plane orthogonal to the longitudinal axial direction;
   a nominal height at a center of the respective lower semicircular base;
   a semi-elliptical upper base surface opposite the respective lower base and inclined with respect to it by a respective internal angle, typically between 8° and 60°.

The two semi-cylindrical prisms 3a and 3b are opposite each other in the height direction along a plane of axial separation so that the respective lower semi-circular bases lie in the same orthogonal plane together composing a full circle.

The known head of FIG. 3 typically has a tubular shank 7 which surrounds the upper base surfaces of the two prisms 3a and 3b, shown in semi-transparency, and is configured to be fitted into a sealing ring of an applanation tonometer while maintaining the cantilevered tonometer head outside the scaling ring, as shown in FIG. 2. The side surface of the current tonometer heads is opaque, so as to shield the prisms 3a and 3b from spurious light rays coming from the side and which could disturb the vision, and notches are made thereon to correctly align the separation plane of the prisms 3a and 3b to the patient.

The patient is made to sit in front of the slit lamp and look at a point of reference, as illustrated in FIG. 4. The slit of the lamp is opened to the maximum and a cobalt blue filter is interposed to better visualize the ophthalmic dye, i.e. to better visualize the edges of the flattened area. A light beam is then directed so that it passes through the transparent prism and the knob is adjusted to the notch corresponding to 10 mmHg. The slit lamp is moved forward so that the prism gently contacts the central part of the cornea, as in FIG. 5.

Prisms 3a and 3b, contained in the transparent plastic head, split the circular image of the flattened corneal surface into two semicircles, one overlapped to the other. Then through the eyepiece, two green semicircles can be observed in a blue light field. FIGS. 6a-6d show what is observed with the Goldmann tonometer in different cases. The lateral knob of the tonometer is rotated so as to bring the two semicircles into contact with their inner edge. Depending on the exerted pressure, different aspects of the semicircles can be observed: if these are far away, it is necessary to increase the applanation force; if they overlap, turn the knob in the opposite direction to decrease the exerted force. In FIG. 6a, the hemicorons appear relatively thick due to excessive use of dye; in FIG. 6b the hemicrowns have an adequate thickness but are not correctly aligned because the applanation force is excessive; in FIG. 6c instead the applanation force is insufficient; in FIG. 6d the hemicrowns are correctly aligned. At this point, the amount of IOP indicated by the value on the knob is determined.

The measurement carried out with the tonometer is influenced by the physical characteristics of the cornea, such as its thickness, its modulus of elasticity, as well as by the attraction caused by the surface tension of the tear film, and in general it does not coincide with the intraocular pressure actually present in the eye, measurable with a manometric technique by inserting a probe through the cornea. According to Goldmann, inventor of the tonometer that bears his name, the effects of these physical characteristics compensate for the attraction due to surface tension when a corneal surface with a diameter of 3.06 mm is flattened. Under such conditions, the pressure measured by the Goldmann tonometer would correspond to the intraocular pressure IOP.

Several scholars have found that this is not strictly true and various formulas have been proposed in the literature to correct the readings provided by the Goldmann tonometer, in order to extrapolate the “true” IOP value. Some of these formulas do not take into account the modulus of elasticity of the cornea; other corrective formulas, very complex to use, are based on a hypothetical standard value of the modulus of elasticity. Unfortunately, the modulus of elasticity of an elderly patient's cornea can even be twice that of a healthy young patient, so corrective formulas may introduce further inaccuracies, rather than improve accuracy.

### SUMMARY

Studies performed by the Applicant have shown that the elastic reaction force exerted by a flattened cornea against the applanation surface of the head of a Goldmann tonometer can be estimated by knowing the modulus of elasticity of the cornea, the central corneal thickness, the radius of the flattened corneal surface and the mean central radius of curvature of the cornea, given by the arithmetic mean between the inner central radius of curvature and the outer central radius of curvature of the cornea. By appropriately shaping the prisms of a Goldmann tonometer head, as indicated in claim 1, it becomes possible to carry out in an accurately repeatable manner two different pressure measurements with two different radii of the flattened corneal surface. From these two different measurements, with a method according to the present disclosure it is possible to obtain the value of the “true” intraocular pressure of the patient, and therefore to correct the classic pressure measurement taken with the Goldmann tonometer. Furthermore, by knowing the central corneal thickness with any pachymeter and an average radius of curvature at the corneal apex with any keratometer, with another method according to the present disclosure it is possible to calculate in vivo the modulus of elasticity of the cornea of a patient according to the two different pressure measurements.

The methods of measuring the intraocular pressure and the modulus of elasticity in vivo are defined in claims 6 and 7.

Other embodiments are defined in the appended claims.

### BRIEF DESCRIPTION OF THE DRAWINGS

FIG. 1 shows a Goldmann tonometer.

FIG. 2 shows a classic head mounted in the holding ring of an applanation tonometer, illuminated by a blue light beam from a slit lamp.

FIG. 3 is a perspective view of a conventional applanation tonometer head.

FIG. 4 shows the familiar Goldmann tonometer head applied to a patient's eye;

FIG. 5 illustrates the principle of measuring intraocular pressure with the Goldmann tonometer.

FIG. 6 shows semicircles or hemicorons visualized with the Goldmann tonometer, in the following cases: a) excessive use of dye; b) excessive applanation force; c) insufficient applanation force; d) correctly aligned hemicorons.

FIG. 7 shows a tonometer head according to the present disclosure, with an open rear tang.

FIG. 8 shows another tonometer head according to the present disclosure, with a rear tang closed by a transparent surface.

FIG. 9 is a sectional view of the tonometer head of FIG. 8 along a longitudinal plane of separation of the two prisms of the head.

FIG. 10 is a perspective view of the tonometer heads of FIGS. 7 and 8 sectioned along the XY plane to highlight the two prisms, each having three upper base surfaces.

FIG. 11 is a plan view of the tonometer heads of FIGS. 7 and 8 sectioned along the XY plane to highlight the two prisms, having straight edges in pairs parallel.

FIG. 12A is a sectional view along the XZ plane of the tonometer head of FIG. 7 with the directions of propagation through the two prisms of the light rays generated by an annulus of ophthalmic dye having a radius equal to g₁, according to a measurement method of intraocular pressure according to this disclosure.

FIG. 12B is a sectional view along the XZ plane of the tonometer head of FIG. 7 with the directions of propagation through the two prisms of the light rays generated by an annulus of ophthalmic dye having a radius equal to g₀, according to the classical method of Goldmann measuring intraocular pressure.

FIG. 13A shows the image that the prisms of a tonometer head of this disclosure form when flattening a circular corneal surface of radius g₁ according to a method of measuring intraocular pressure according to this disclosure.

FIG. 13B shows the image that the prisms of a tonometer head of this disclosure form when a circular corneal surface of radius g₀ is flattened according to Goldmann's classic method of measuring intraocular pressure.

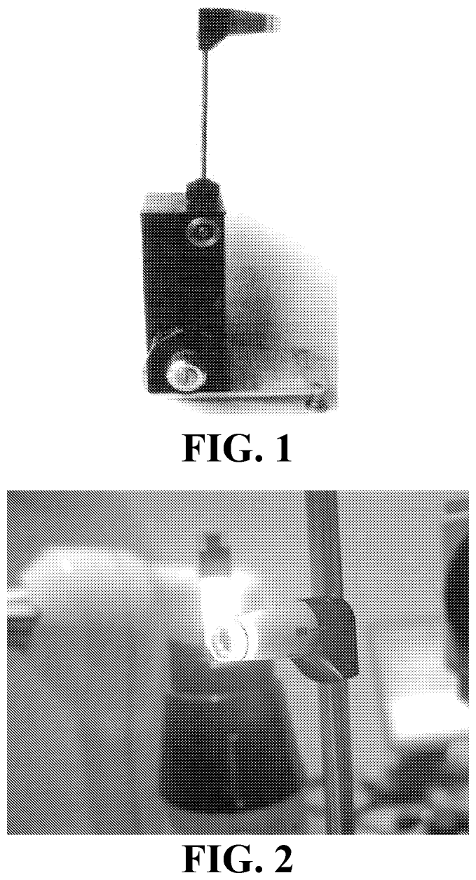

### DESCRIPTION OF EXEMPLARY EMBODIMENTS

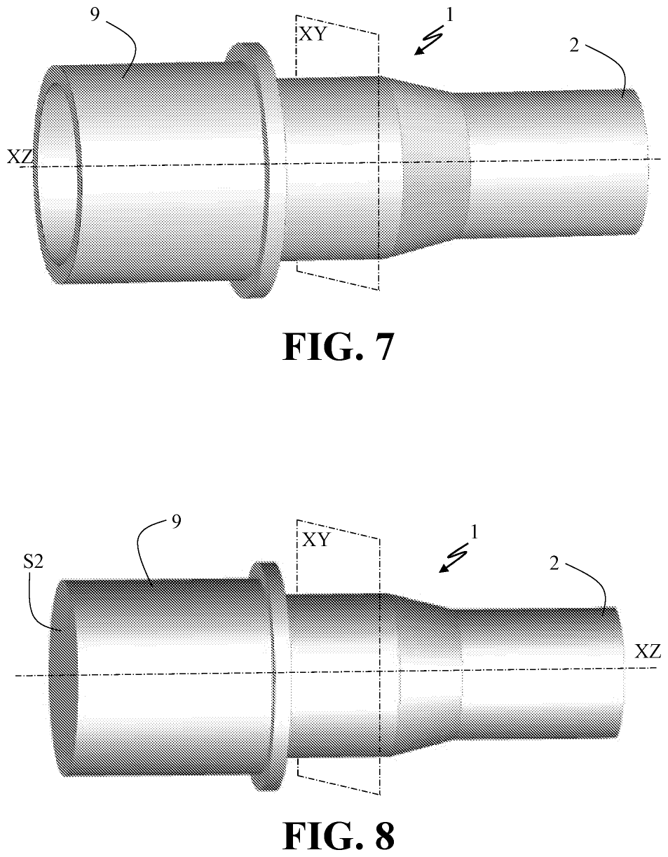

Some heads 1 for applanation tonometers according to the present disclosure are shown in FIG. 7, with rear tang 9 open, and in FIG. 8, with rear tang 9 closed by a transparent surface S2.

The embodiment of FIG. 7 can be more easily made by injection molding of transparent plastic material, such as for example PMMA or ABS, since it does not provide any closed cavity. Typically, disposable-type tonometer heads are made this way because they can be made in a single thermoplastic molding operation. Otherwise, the embodiment of FIG. 8 will be preferable for reusable tonometer heads, for example made of glass, which conveniently do not have open cavities, which are difficult to sterilize and dry, but have a transparent rear cover S2 which closes the internal cavity sealing it and preventing liquids or dust from entering it.

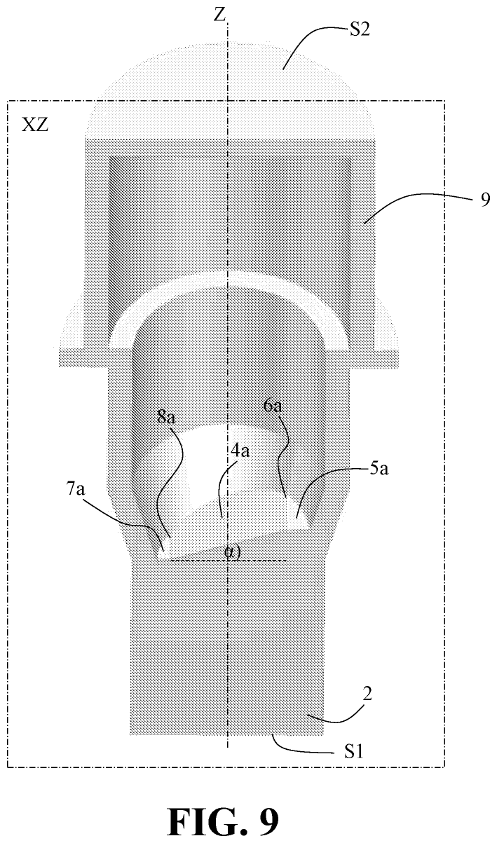

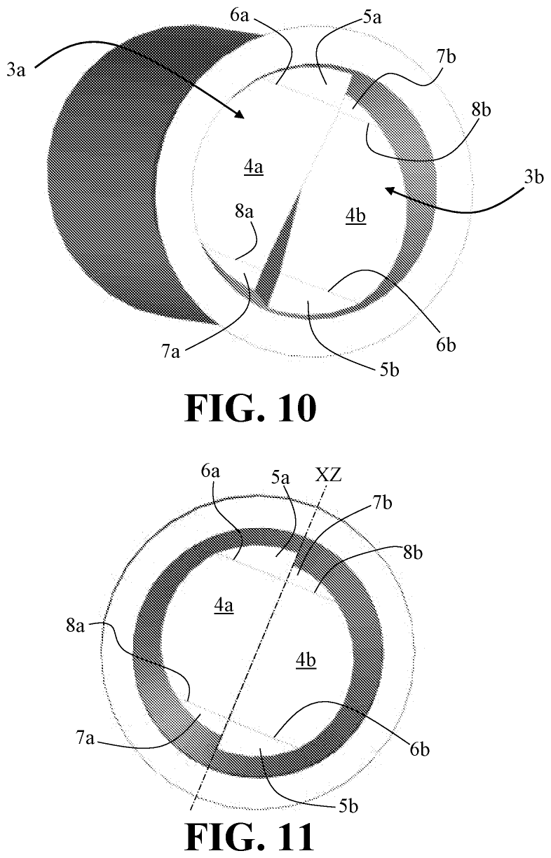

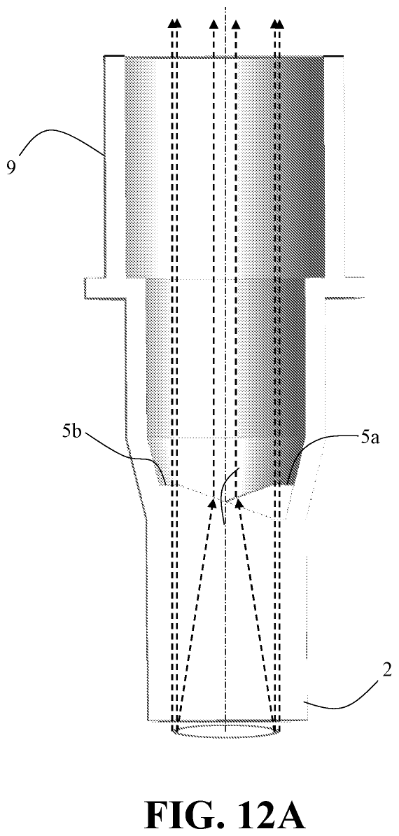

Like common applanation tonometer heads, a head 1 according to the present disclosure has a transparent body having a longitudinal axis Z, defining, at a first end along the longitudinal axis Z, an end portion 2 which has a surface circular or elliptical corneal applanation plane S1, having a respective radius or semi-axis of applanation, arranged on a first base plane of the end portion 2 orthogonal to the longitudinal axis Z of the tonometer head. In the central part of the head 1, two semi-cylindrical or semi-elliptical prisms 3a and 3b are defined, visible in FIGS. 10 and 11 which represent, respectively in perspective and in plan view, a section of the head of FIG. 7 or of FIG. 8 along the XY plane. The two prisms 3a, 3b are integral with each other and with the end portion 2 and each of them has:
a respective lower semicircular or semielliptical base integral with said first end and arranged along a second plane orthogonal to the longitudinal axis Z and parallel to the flat corneal applanation surface S1;
a first semi-elliptical upper base surface 4a opposite the respective lower base and inclined with respect to the lower base by a respective internal angle α comprised between 9° and 60°, highlighted in the sectional view of FIG. 9 along the plane XZ.

As shown in FIGS. 10 and 11, the two semi-cylindrical or semi-elliptical prisms 3a, 3b are opposite along the longitudinal axis with respect to the axial separation plane XZ so that the respective lower semi-circular or semi-elliptical bases are coplanar and lie in the second plane orthogonal by composing together a filled circle or a filled ellipse, respectively.

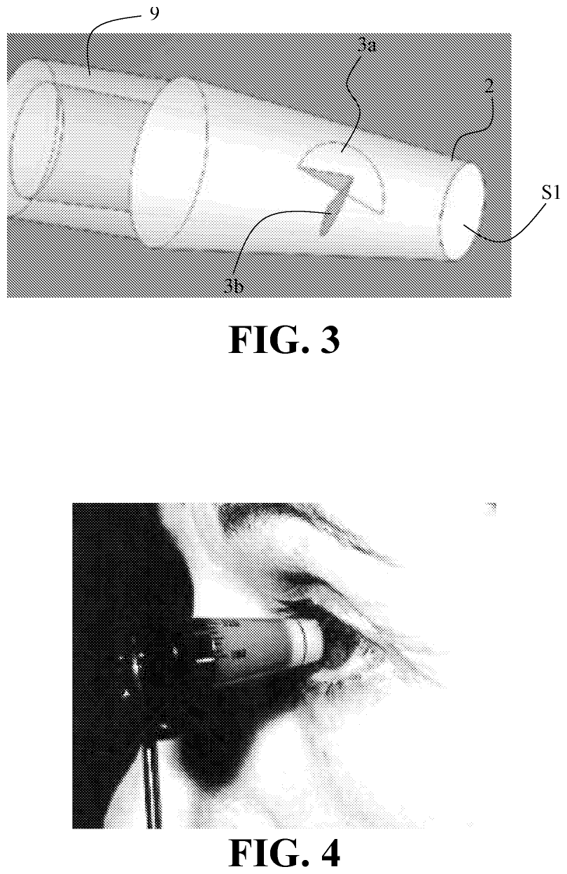

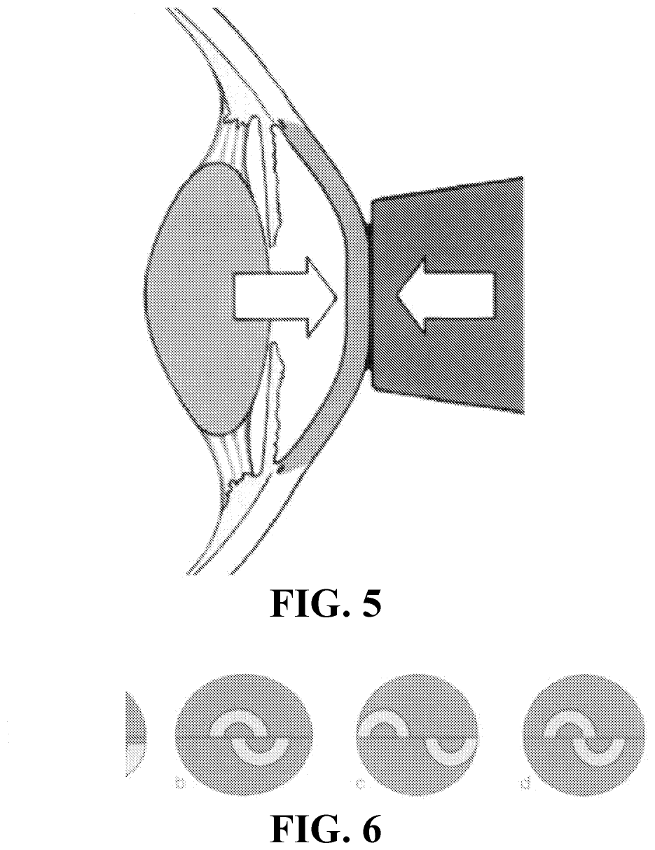

A particularity of the tonometer heads 1 of this disclosure consists in the fact that each of the two prisms is not defined only by an inclined upper surface 4a, as in the prisms of the classic head of FIG. 3, but has a profile in the shape of a broken line, clearly visible in FIG. 9. More specifically, each of the two prisms 3a, 3b defines at least a second upper base surface 5a, 5b parallel to the flat circular or elliptical corneal applanation surface S1 and bordering the first upper base surface 4a, 4b without gaps at a respective first straight edge 6a, 6b, which is at a first distance (measured in the axial separation plane XZ) from the longitudinal axis Z.

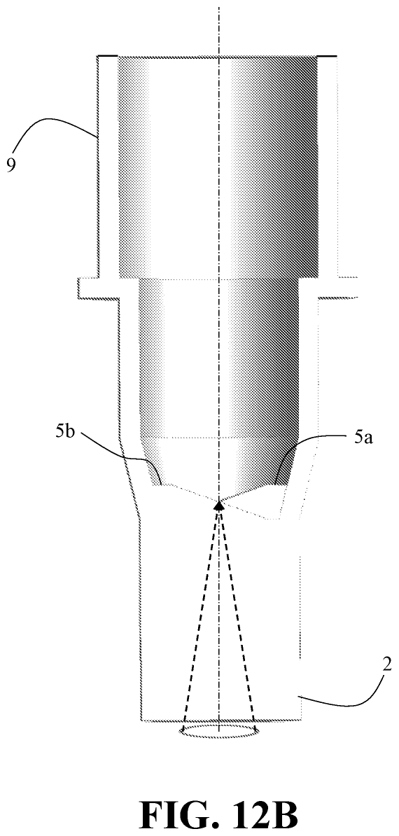

In practice, each of the two prisms 3a and 3b has at least a first inclined upper base surface 4a, 4b and a second upper base surface 5a, 5b parallel to the applanation surface S1. Thanks to this configuration of the second upper base surface 5a, 5b, a ray of light entering the end portion 2 from a point of the flat circular or elliptical corneal applanation surface S1 at a distance from the longitudinal axis Z equal to or greater than the first distance and which is directed along the axial separation plane XZ parallel to the longitudinal axis Z, crosses the end portion 2 and protrudes from the second upper base surface 5a always remaining directed parallel to the longitudinal axis, as schematically shown in FIG. 12A.

Each first straight edge 6a, 6b is at a first distance from the longitudinal axis Z and this first distance is between 1.6 mm and 3 mm. As will appear more clearly below, the presence of the first straight edge 6a, 6b and the value of this first distance are important to allow an accurate measurement of the intraocular pressure and to allow an in vivo estimate of the value of the modulus of elasticity E of the cornea of a patient.

FIGS. 9 to 14 refer to a preferred configuration in which each of the two prisms 3a, 3b, in addition to having a first inclined upper base surface 4a, 4b and a second upper base surface 5a, 5b parallel to the applanation surface S1, also has an optional third upper base surface 7a, 7b, parallel to the flat circular or elliptical corneal applanation surface S1, and bordering the first upper base surface 4a, 4b without gaps at a second respective straight edge 8a, 8b parallel to the first straight edge 6a, 6b and positioned on an opposite side with respect to the respective first upper base surface 4a, 4b.

As can be clearly seen in FIG. 11, also the second straight edge 8a, 8b is at the first distance from the longitudinal axis so that, when the head 1 is viewed exactly in the axial direction Z, the first straight edge 6a, 6b of each prism 3a, 3b appears as the extension of the second straight edge 8b, 8a of the other prism 3b, 3a. When this condition occurs, the ophthalmologist knows that he is looking through the head 1 of the tonometer in a perfectly axial direction, so that it is in the right conditions to carry out the pressure measurements.

As well as the second upper base surface 6a, 6b, also the optional third upper base surface 8a, 8b is configured so that a ray of light entering the end portion 2 from a point on the flat circular or elliptical surface of corneal applanation S1 at a distance from the longitudinal axis Z equal to or greater than the first distance and which is directed along the axial separation plane XZ parallel to the longitudinal axis Z, crosses the end portion 2 and protrudes from the third upper base surface 11c always remaining directed parallel to the longitudinal axis.

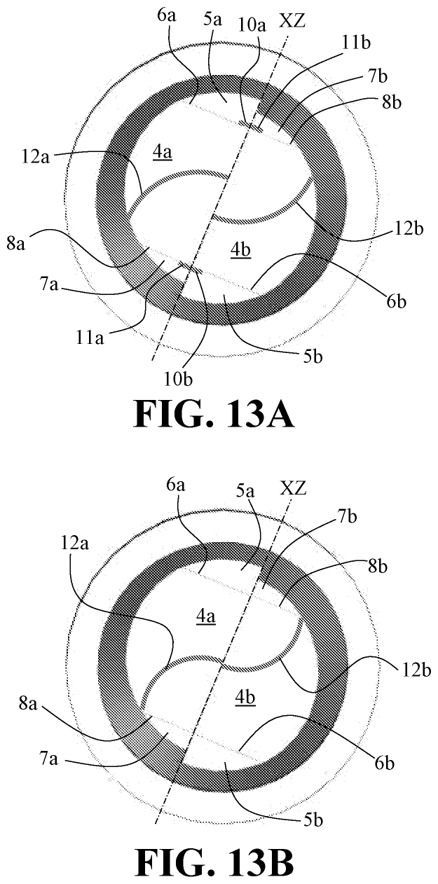

The head 1 according to the present disclosure allows two pressure measurements to be carried out with great accuracy with the Goldmann tonometer: a first measurement is carried out by smoothing a corneal surface of radius g₁ equal to the first distance of the edges 6a, 6b from the longitudinal axis, as shown in FIG. 12A, and a second measurement is performed in the classic Goldmann method, as shown in FIG. 12B, by smoothing a corneal surface of radius g₀ equal to 1.53 mm. When the first measurement is carried out (FIG. 12A), the upper surfaces of the two prisms 3a and 3b divide the luminous contour of the flattened corneal surface of radius g₁ in the manner illustrated in FIG. 13A. This image is obtained only if the flattened corneal surface of radius g₁ is perfectly centered with respect to the longitudinal Z axis of the head. In fact, only in this condition, two first light arcs 10a and 10b are formed on the surfaces 5a and 5b of the two prisms 3a and 3b, the light rays of the two arcs enter (FIG. 12A) in the end portion 2 from the flat corneal applanation surface S1 at a distance from the longitudinal axis Z equal to or greater than the first distance, they propagate parallel to the longitudinal axis Z and emerge from the second upper base surface 5a, 5b always remaining directed parallel to the longitudinal axis Z.

FIGS. 12A and 13A also show the optional third upper base surface 7a, 7b, also parallel to the flat corneal applanation surface S1, with the related second edge 8a, 8b at the same first distance from the longitudinal axis Z, exactly like the first edge 6a, 6b. Thanks to this optional expedient, two second light arcs 11a and 11b are formed on the third upper base surfaces 7a and 7b of the two prisms 3a and 3b in a completely analogous way to what occurs for the first light arcs 10a and 10b. This optional embodiment is convenient because it allows the centering of the longitudinal axis Z with respect to the flattened corneal surface to be made even more accurate. Indeed, only with perfect centering will the first light arc 10a coming out of the first prism 3a appear as an extension of the second light arc 11b coming out of the second prism 3b, and at the same time the first light arc 10b coming out of the second prism 3b will appear as the extension of the second luminous arc 11a which emerges from the first prism 3a, as shown in FIG. 13A. Through the first upper base surfaces 4a and 4b two hemicorons 12a and 12b will be seen which do not touch each other, as in FIG. 6b, because the radius of the flattened corneal surface g₁ is greater than the radius g₀ for the measurement of the intraocular pressure according to the method of Goldmann.

On the other hand, when the second measurement (FIG. 12B) is carried out in the classic Goldmann way, the two hemicrons 12a and 12b join in the classic way illustrated in FIG. 13B. If the flattened corneal surface is centered with respect to the longitudinal axis Z, the arcs through the surfaces 5a, 5b, 7a, 7b will not be seen because the radius g₀ is smaller than the first distance of the edges 6a, 6b, 8a, 8b.

An advantage of the tonometer head according to the present disclosure is that it allows for accurate centering of the applanation surface with respect to the apex of the cornea. Currently, the ophthalmologist who carries out the measurement with the Goldmann tonometer believes that the tonometer head is correctly positioned with respect to the corneal apex when the point of contact between the two hemicorons (FIG. 13B) is more or less in the center of the field of vision. However, this adjustment is approximate and can introduce further errors due to incorrect positioning.

With the tonometer head of the present disclosure, it is first possible to measure the pressure P_{G1} by flattening a corneal surface with a radius equal to g₁, until the mirrored arcs 10a and 10b appear with respect to the axial separation plane XZ, which can be performed only if the tonometer head is perfectly centered with respect to the corneal apex, and then the Poo pressure is measured in the classic way by simply reducing the force exerted by the Goldmann tonometer on the cornea without moving it with respect to the previous position.

According to one aspect, the radius g₁, i.e. the distance from each of the edges 6a, 6b to the longitudinal axis Z measured along the axial separation plane XZ, is between 1.6 mm and 3 mm, preferably between 1.8 mm and 2.6 mm. Preferably, this radius g₁ is equal to 2.0 mm.

In one aspect, the flat corneal applanation surface S1 is circular and has a radius ranging between 3.0 mm and 4.6 mm.

With the applanation head of this disclosure it is possible to carry out a first pressure measurement with the Goldmann tonometer by flattening a corneal surface with a radius equal to g₁ (FIG. 12A), as well as a second standard pressure measurement by smoothing a corneal surface with a radius g₀ (smaller than the radius g₁) according to the classic Goldmann method. In both measurements, the ophthalmologist has visual reference points which allow him to establish with great accuracy when it is necessary to read the pressure indication on the knob placed on the tonometer.

The possibility of carrying out a pressure measurement when the flattened corneal surface has a radius g₁ greater than g₀ is an aspect that makes it possible to estimate the corneal elasticity modulus E in vivo, as well as to correct the standard pressure measurement according to the classic Goldmann method removing the disturbance caused by greater or lesser corneal stiffness, thus calculating the “true” intraocular pressure Prop. In order to understand how the methods of the present disclosure allow these excellent results to be achieved with the tonometer head of this disclosure, the results of mathematical studies carried out by the Applicant on the elastic deformation of spherical shells of constant thickness are reported.

Even if globally the cornea is not comparable to a spherical shell of constant thickness, the flattened part during the measurement of the intraocular pressure with the Goldmann tonometer is sufficiently small to be approximated to a spherical shell of practically constant thickness. Theoretical studies carried out by the Applicant on the deformations of elastic spherical shells of constant thickness have shown that the distribution of the pressure exerted by a flattened cornea can be calculated using principles of theory of elastic deformations and that it depends on the central corneal thickness, the central mean radius of curvature of the cornea before applanation, the diameter of the flattened corneal surface, the intraocular fluid pressure (the IOP), and the coefficient of elasticity of the cornea. These theoretical results have been confirmed by laboratory tests carried out by the applicant.

Studies carried out by the Applicant have shown that the overall average pressure exerted by the elastic reaction of the flattened cornea against the head of the Goldmann tonometer alone can be expressed as a function of the central corneal thickness, the central mean radius of curvature of the cornea before applanation, the radius of the flattened corneal surface and the coefficient of elasticity of the cornea. Without thereby being bound by any theory, the Applicant has noted that this average pressure can be approximated as follows:

$$\frac{E \cdot h \cdot {g}^{2}}{{R}^{3}} \qquad ( 1 )$$

in which
E=Young's modulus averaged over corneal thickness;
h=apical corneal thickness, at the center of the cornea;
g=radius of the flattened corneal area;
R=average radius of curvature (inner radius plus outer radius, divided by two) of the cornea measured in the apical area, i.e. at the center of the cornea.

In the following description, reference will be made to formula (1) for the calculation of the elastic reaction of the flattened cornea only, however what will be said hereinafter remains valid mutatis mutandis even if the corneal elastic reaction is estimated with a different formula.

Goldmann established standard reference conditions of central corneal thickness h₀, (mean) radius of curvature R₀ and modulus of elasticity (Young's modulus) E₀ for which the pressure P₀ measured on the shaft of his tonometer accurately matched the intraocular pressure Prop. It is commonly believed that the Goldmann tonometer measures intraocular pressure fairly accurately when the cornea has:
a central corneal thickness h₀ equal to 0.536 mm;
a modulus of elasticity E₀ (Young's modulus) equal to 0.19 MPa;
an external corneal radius of curvature at the center equal to 7.6 mm;
an internal corneal radius of curvature at the center equal to 6.7 mm.

This is equivalent to saying that:
E₀=0.19 MPa;
h₀=0.536 mm;
g₀=1.53 mm is the radius of the applanation area of the Goldmann tonometer;
R₀=7.15 mm is the mean radius of curvature.

Since the overall average pressure exerted by the elastic reaction of the flattened cornea against the head of the Goldmann tonometer alone, can be estimated approximately with the following ratio:

$$\frac{E \cdot h \cdot \text{?}}{\text{?}} \qquad ( 2 )$$

$$\text{?} \text{indicates text missing or illegible when filed}$$

then it means that the pressure P₀ measured on the rod of the Goldmann tonometer according to the classic Goldmann method, is approximately given by:

$$\text{?} ≅ \text{?} + \frac{E \cdot h \cdot \text{?}}{\text{?}} - \frac{\text{?} \cdot \text{?} \cdot \text{?}}{\text{?}} \qquad ( 3 )$$

$$\text{?} \text{indicates text missing or illegible when filed}$$

With a tonometer head according to the present disclosure, a new pressure measurement, hereinafter referred to as Poi, can be accurately made by flattening a corneal surface having a radius g₁ greater than the radius g₀ determined by the classic Goldmann method. This radius g₁ is equal to half of the reciprocal distance between the straight edges 6a and 6b respectively of the first prism 3a and of the second prism 3b so that, when on the second upper base surface 5a, 5b of each of the two prisms it is possible to see the inner edge of the two hemicrowns (FIG. 12A), then the longitudinal axis of the tonometer head is perfectly aligned with the corneal apex, because the flattened corneal surface is centered with respect to the corneal apex and has a radius equal to g₁. In this new measurement condition, the new measured pressure Poi can be approximated as follows:

$$\text{?} ≅ \frac{\text{?}}{\text{?}} \text{?} + \frac{E \cdot h \cdot \text{?}}{\text{?} \cdot \text{?}} - \frac{\text{?} \cdot \text{?} \cdot \text{?}}{\text{?}} \qquad ( 4 )$$

$$\text{?} \text{indicates text missing or illegible when filed}$$

Combining eq. (4) with eq. (3) it is possible to know the value of the “true” Prop pressure and the value of the corneal modulus of elasticity E:

$$\frac{E \cdot h}{\text{?}} ≅ \frac{1}{\text{?} - \text{?}} \cdot [ \frac{\text{?} \cdot \text{?} \cdot \text{?}}{\text{?}} \cdot ( \frac{\text{?}}{\text{?}} - 1 ) + \frac{\text{?}}{\text{?}} \cdot \text{?} - \text{?} ] \qquad ( 5 )$$

$$\text{?} ≅ \frac{1}{\text{?} - \text{?}} \cdot [ \text{?} \cdot \text{?} - \frac{\text{?}}{\text{?}} \cdot \text{?} ] + \frac{\text{?} \cdot \text{?} \cdot \text{?}}{\text{?}} \cdot ( \frac{\text{?}}{\text{?}} + 1 ) \qquad ( 6 )$$

$$\text{?} \text{indicates text missing or illegible when filed}$$

By measuring the central corneal thickness h using any corneal pachymeter and using any keratometer the mean central corneal curvature radius R, knowing the radius g₀ of the applanation surface according to the classic method (it is always equal to 1.53 mm) and knowing the radius g₁ of the corneal surface flattened with the new measurement according to the present disclosure, the corneal modulus of elasticity E can be easily calculated.

Even if the exact values of the corneal thickness and the mean apical radius of curvature of the cornea are not known, with the aim of knowing the value of the modulus of elasticity only approximately, one can use, instead of the true “h” value and the true “R” value, also corresponding estimated values of them, obtaining in any case an in vivo and reasonably accurate estimate of the value of the modulus of elasticity E. For example, the estimated values for “h” and for “R” may be respective values standard of central corneal thickness h₀ and (average) radius of curvature R₀ of a typical cornea.

With the applanation head for a Goldmann tonometer according to this disclosure, it is possible to measure the elastic reaction of a flattened cornea by detecting the pressures P_{G0} and P_{G1} measured by the Goldmann tonometer respectively when the cornea is flattened with a radius g₀ (classical measurement). and with a radius g₁ (new measurement).

Thanks to the double pressure measurement, which may be performed accurately using the tonometer head of this disclosure, it is possible to correct the pressure measurement P_{G0} taken by the Goldmann tonometer according to the classical technique to obtain the “true” Prop intraocular pressure.

From equation (6) it can be deduced that, by also carrying out the Poi pressure measurement, it is possible to correct the value of the Poo pressure measurement detected by the Goldmann tonometer with the classic technique, to calculate the “true” P_{IOP} intraocular pressure of a patient:
without knowing the average curvature radius R of the cornea;
without knowing the modulus of elasticity E of the cornea;
without knowing the central corneal thickness h.

Consequently, thanks to the tonometer head of the present disclosure it is possible to calculate the “true” intraocular pressure Prop of a patient without having to use a pachymeter and a keratometer and without resorting to the formulas known in the literature currently used to correct the pressure measurement P_{G0} detected by Goldmann's tonometer, which were obtained heuristically.

Having at one's disposal a Goldmann applanation tonometer and a tonometer head according to the present disclosure, the “true” intraocular pressure Prop of a patient's eye can be measured by the following operations:
administering an eye dye to a patient's cornea;
detecting, according to Goldmann's classical measurement method, a total pressure P_{G0} exerted by said cornea pressed against the applanation surface of the tonometer head;
detecting a new total pressure P_{G1} exerted by the cornea pressed against the applanation surface of the tonometer head when a circular corneal surface of radius equal to the radius g₁ is flattened against the applanation surface S1 of the tonometer head;
calculating the intraocular pressure of the eye on the basis of the pressure P_{G0} and the pressure P_{G1}. The latter operation can be carried out for example by using equation (6).

Conveniently, the operations for detecting the new pressure P_{G1} and the pressure P_{G0} can be reversed, so as to accurately center the tonometer head with respect to the corneal apex, before measuring the pressure Poo according to the classic measurement method by Goldmann.

To measure the modulus of elasticity E of a cornea of a patient's eye in vivo using a Goldmann applanation tonometer and a tonometer head according to the present disclosure, the following steps are performed:
measuring a central corneal thickness h and a central curvature radius R of the cornea of the patient's eye;
administering an eye dye to the cornea;
detecting, according to Goldmann's classical measurement method, a total pressure P_{G0} exerted by said cornea pressed against the applanation surface of the tonometer head;
detecting a new overall pressure Poi exerted by the cornea pressed against the applanation surface of the tonometer head when a circular corneal surface of radius equal to the radius g₁ is flattened against the applanation surface S1 of the tonometer head;
calculating the corneal modulus of elasticity based on the first pressure P_{G0}, on the new pressure Poi, on the central corneal thickness h and on the central curvature radius R of the patient's cornea.

According to an aspect not shown in the figures, the second distance g₂ of the second straight edges 8a, 8b from the longitudinal axis Z, measured in the axial separation plane XZ, is different from the first distance g₁ of the straight edges 6a, 6b, so that the second upper base surfaces 5a, 5b are more distant from the longitudinal axis Z than the third upper base surfaces 7a, 7b. According to one aspect, the opposite is also possible, i.e. that the first distance g₁ be greater than the second distance g₂.

According to one aspect, the radius g₂, i.e. the distance from each of the edges 8a, 8b to the longitudinal axis Z measured along the axial separation plane XZ, is between 1.6 mm and 3 mm, for example between 1.8 mm and 2.6 mm. For example, this radius g₂ is equal to 2.5 mm.

According to one aspect, the radius g₂ is equal to 2.0 mm and the radius g₁ is equal to 2.5 mm. With these values it has been experimentally noted that the estimates obtained for the parameters E₀, E and Prop are less sensitive to imprecisions in the detection of the pressures P_{G0}, P_{G1} and P_{G2}.

In general, by ensuring that the two distances g₁ and g₂ are different, it is possible to carry out a third pressure measurement in the same way as shown in FIG. 12A. If, for example, the second distance g₂ is greater than the first distance g₁, then it will be necessary to flatten a corneal surface with a radius equal to at least g₂ (greater than g₁) in order to see a luminous arc respectively through the third upper base surfaces 7a and 7b: when the inner edge of these luminous arcs touches the respective second straight edge 8a and 8b, then the radius of the flattened corneal surface is equal to g₂. In this case, the pressure P_{G2} will be given by the following equation:

$$\text{?} ≅ \frac{\text{?}}{\text{?}} \text{?} + \frac{E \cdot h \cdot \text{?}}{\text{?} \cdot \text{?}} - \frac{\text{?} \cdot \text{?} \cdot \text{?}}{\text{?}} \qquad ( 7 )$$

$$\text{?} \text{indicates text missing or illegible when filed}$$

This equation, taken together with equations (3) and (4):

$$\text{?} ≅ \frac{\text{?}}{\text{?}} \text{?} + \frac{E \cdot h \cdot \text{?}}{\text{?} \cdot \text{?}} - \frac{\text{?} \cdot \text{?} \cdot \text{?}}{\text{?}} \qquad ( 4 )$$

$$\text{?} ≅ \text{?} + \frac{E \cdot h \cdot \text{?}}{\text{?}} - \frac{\text{?} \cdot \text{?} \cdot \text{?}}{\text{?}} \qquad ( 3 )$$

$$\text{?} \text{indicates text missing or illegible when filed}$$

allows to calculate the “true” intraocular pressure P_{IOP}:

$$\text{?} ≅ \frac{\text{?} \cdot [ \text{?} \cdot ( \text{?} - \text{?} ) + \text{?} \cdot ( \text{?} - \text{?} ) + \text{?} \cdot ( \text{?} - \text{?} ) ]}{\text{?} \cdot \text{?} + \text{?} \cdot \text{?} + \text{?} \cdot \text{?} - \text{?} \cdot \text{?} - \text{?} \cdot \text{?} - \text{?} \cdot \text{?}} \qquad ( 8 )$$

$$\text{?} \text{indicates text missing or illegible when filed}$$

the modulus of elasticity E:

$$E ≅ \frac{\text{?}}{h} \cdot \frac{\text{?} \cdot [ \text{?} \cdot ( \text{?} - \text{?} ) + \text{?} \cdot ( \text{?} - \text{?} ) + \text{?} \cdot ( \text{?} - \text{?} ) ]}{\text{?} \cdot \text{?} + \text{?} \cdot \text{?} + \text{?} \cdot \text{?} - \text{?} \cdot \text{?} - \text{?} \cdot \text{?} - \text{?} \cdot \text{?}} \qquad ( 9 )$$

$$\text{?} \text{indicates text missing or illegible when filed}$$

as well as the reference modulus of elasticity E₀ of the Goldmann tonometer:

$$\text{?} ≅ \frac{\text{?}}{\text{?} \cdot \text{?}} \cdot \frac{\begin{matrix}\text{?} \cdot ( \text{?} \cdot \text{?} - \text{?} \cdot \text{?} ) + \text{?} \cdot ( \text{?} \cdot \text{?} - \text{?} \cdot \text{?} ) + \\ \text{?} \cdot ( \text{?} \cdot \text{?} - \text{?} \cdot \text{?} )\end{matrix}}{\text{?} \cdot \text{?} + \text{?} \cdot \text{?} + \text{?} \cdot \text{?} - \text{?} \cdot \text{?} - \text{?} \cdot \text{?} - \text{?} \cdot \text{?}} \qquad ( 10 )$$

$$\text{?} \text{indicates text missing or illegible when filed}$$

Accurately knowing the value of the reference modulus of elasticity E₀ of a Goldmann tonometer makes it possible to accurately calculate the value of the modulus of elasticity E of a patient's cornea, using the same Goldmann tonometer and a head of the type shown in figures from 7 to 13B, wherein the first edges 6a, 6b are at the same distance as the second edges 8a, 8b from the longitudinal axis Z. Thanks to a tonometer head according to this disclosure with second straight edges 8a, 8b at a distance g₂ and first straight edges 6a, 6b at distance g₁ from the longitudinal axis Z, eq. (10) allows to check if the reference E₀ modulus value of the Goldmann tonometer (on which the head is mounted) has remained the same as the factory value or if it has changed, requiring an operation of calibration of the tonometer. A calibration head can therefore be used with second straight edges 8a, 8b at a distance g₂ and first straight edges 6a, 6b at a distance g₁ from the longitudinal axis Z, for the preliminary determination of the reference modulus of elasticity E₀ of the Goldmann tonometer, in order to then be able to measure more easily the modulus of elasticity E of a patient's cornea by using the head of this disclosure with straight edges 6a, 6b, 8a, 8b equidistant from the longitudinal axis Z.

1. A head for applanation tonometer, comprising a transparent body having a longitudinal axis, defining:
at a first end along said longitudinal axis, an end portion defining a flat circular or elliptical corneal applanation surface, having a respective radius or semi-axis of applanation, arranged on a first plane of base of the end portion orthogonal to the longitudinal axis of the tonometer head;
two semi-cylindrical or semi-elliptical prisms, each having:
a respective lower semicircular or semi-elliptical base integral with said first end and arranged along a second plane orthogonal to said longitudinal axis and parallel to the flat corneal applanation surface;
a first semi-elliptical upper base surface opposite said respective lower base and inclined with respect to the lower base by a respective internal angle between 8° and 60°;
said two semi-cylindrical or semi-elliptical prisms being opposite along said longitudinal axis with respect to an axial separation plane so that the respective lower semicircular or semi-elliptic bases lie on said second orthogonal plane composing together a full circle or a full ellipse, respectively;
said end portion and said two prisms are integral with each other; wherein
each of said two prisms defines at least a second upper base surface parallel to said flat circular or elliptical corneal applanation surface and bordering said first upper base surface without interruption at a first straight edge,
wherein said first straight edge is at a first distance from said longitudinal axis and said first distance is between 1.6 mm and 3 mm,
wherein said second upper base surface is configured so that a ray of light entering said end portion from a point of the flat circular or elliptical corneal applanation surface at a distance from the longitudinal axis equal to or greater than said first distance and which is directed parallel to said longitudinal axis along said axial separation plane, crosses said end portion and exits from said second surface of the upper base always remaining directed parallel to said longitudinal axis.

2. The head for applanation tonometer according to claim 1, wherein:
at least one of said two prisms defines at least a third upper base surface parallel to said flat circular or elliptical corneal applanation surface and bordering said first upper base surface without interruption at a second straight edge parallel to said first straight edge and positioned on an opposite side with respect to said respective first upper base surface,
said second straight edge is at a second distance from said longitudinal axis,
said third upper base surface is configured so that a ray of light entering
said end portion from a point of the flat circular or elliptical corneal applanation surface at a distance from longitudinal axis equal to or greater than said second distance and which is directed parallel to said longitudinal axis along said axial separation plane, passes through said end portion and exits from said third base surface upper always remaining directed parallel to said longitudinal axis.

3. The head for applanation tonometer according to claim 2, wherein said second distance is equal to said first distance.

4. The head for applanation tonometer according to claim 2, wherein said first distance and said second distance are between 1.8 mm and 2.6 mm.

5. The head for applanation tonometer according to claim 4, wherein said first distance or said second distance is equal to 2.0 mm and said second distance or said first distance is equal to 2.5 mm.

6. The head for applanation according to claim 1, wherein said corneal applanation flat surface is circular and has a radius between 3.0 mm and 4.6 mm.

7. A method of measuring an intraocular pressure of a patient's eye,
comprising the following operations:
providing a Goldmann applanation tonometer;
providing and installing a tonometer head according to claim 1;
administering an ophthalmic dye to a patient's cornea;
detecting, according to a Goldmann method of measuring an intraocular pressure, a first overall pressure exerted by said cornea flattened against the applanation surface of the tonometer head;
detecting a second overall pressure exerted by the cornea flattened against the applanation surface of the tonometer head when a circular corneal surface having a radius equal to said first distance is flattened against the applanation surface; and
calculating the intraocular pressure of the eye on the basis of said first pressure and of said second pressure.

8. A method of in vivo measurement of a modulus of elasticity of a cornea of a patient's eye, comprising the following operations:
providing a Goldmann applanation tonometer;
providing and installing a tonometer head according to claim 1;
measuring a central corneal thickness and a central curvature radius of the cornea of said patient's eye;
administering an ophthalmic dye on said cornea;
detecting, according to a Goldmann method of measuring intraocular pressure, a first overall pressure exerted by the cornea pressed against the applanation surface of the tonometer head;
detecting a second overall pressure exerted by the cornea flattened against the applanation surface of the tonometer head when a circular corneal surface having a radius equal to said first distance is flattened against the applanation surface; and
calculating the modulus of elasticity of the cornea on the basis of said first pressure, of said second pressure, of said central corneal thickness and of said central radius of curvature.

9. The method according to claim 8, comprising the step of determining said central corneal radius of curvature as an intermediate value between its inner central radius of curvature and its outer central radius of curvature.

10. The method according to claim 8, comprising the operations of:
providing and installing a tonometer head;
detecting a third overall pressure exerted by the cornea pressed against the applanation surface of the tonometer head when a circular corneal surface having a radius equal to said second distance is flattened against the applanation surface; and
calculating the modulus of elasticity of the cornea on the basis of said first pressure, of said second pressure, of said third pressure, of said central corneal thickness and of said central radius of curvature.
